(19) United States
(12) Patent Application Publication
(10) Pub. No.: US 2017/0303866 A1
(43) Pub. Date: Oct. 26, 2017

(54) Medical Alert System

(71) Applicant: Thomas A. Hatzilabrou, Chicago, IL (US)

(72) Inventor: Thomas A. Hatzilabrou, Chicago, IL (US)

(21) Appl. No.: 15/494,500

(22) Filed: Apr. 22, 2017

Related U.S. Application Data
(60) Provisional application No. 62/326,349, filed on Apr. 22, 2016.

(51) Int. Cl. A61B 5/00; A61B 5/024

(52) U.S. Cl. CPC A61B 5/746; A61B 5/681; A61B 5/0022; A61B 5/02438

(57) ABSTRACT

A medical alert system comprises a wearable heart monitoring device for transmitting wireless heart rate data signals to a portable computing device. A processor associated with the portable computing device processes the heart rate data signals to determine whether the signals meet pre-established triggering criteria. A transmitter associated with the portable computing device transmits the triggering heart rate data signals to a wireless remote receiver/transmitter connected to the Internet. A receiver, which is associated with one or both of (i) an emergency medical service provider, or (ii) one or more recipient from among a list of designated emergency contacts, receives wireless/cellular transmissions via a portable computing device. In operation, receipt of the triggering heart rate data signals by one or both of the emergency service provider or the recipient(s) provides information sufficient to determine whether an abnormality of sufficient magnitude exists to warrant an emergency medical service response to the user.

### CROSS-REFERENCE TO RELATED APPLICATION

The present application claims priority benefits from U.S. provisional patent application Ser. No. 62/326,349 filed on Apr. 22, 2016, entitled “System and Application Interface with Wearable Heart Rate Monitoring for Emergency Conditions”. The '349 provisional application is hereby incorporated by reference herein in its entirety.

### FIELD OF INVENTION

The present invention relates to cardiac monitoring, and more particularly to a wearable heart rate monitor that has wireless alert capabilities.

### BACKGROUND OF THE INVENTION

A person experiencing a heart attack, or some other disabling cardiac event, can become incapacitated to the point of being unable to request assistance, even to the point of not being able to press a panic button or activate some other triggering device associated with a medical alert system. Persons experiencing a cardiac event during sleep could also be compromised in their ability to seek or send for help, and could even be unaware that they are undergoing a disabling cardiac event. Moreover, persons with inherently abnormal or erratic heart rate patterns may not perceive an oncoming disabling cardiac event.

In a similar manner, persons undergoing a disabling choking experience involving potential suffocation can become incapable of communicating their distress. In other circumstances, persons can become unresponsive, or even comatose, due to disabling events associated with diabetes, epilepsy and other incapacitating medical conditions. In these circumstances, as with incapacitating cardiac events, the person incapacitated could become unable to request assistance or activate some other triggering device associated with a medical alert system.

Existing medical alert systems require at-risk individuals to first make a determination of their distressed condition before taking action to secure help. Moreover, existing medical alert systems do not provide for the transmission of audible or printed/text messages from a user device that would apprise responding personnel of known or suspected pre-existing conditions that could lead to medically harmful or life-threatening consequences. Existing medical alert systems are thus incapable of automatically activating emergency response systems without depending upon the ability of the user being aware of their potentially debilitating condition. In short, existing systems are incapable of making decisions automatically, upon the receipt of signals indicative of a debilitating event, so that help can be summoned without human intervention.

### SUMMARY OF THE INVENTION

The above and other shortcomings of existing medical alert systems are overcome by a system comprising:
(a) a heart monitoring device wearable by a user, the wearable heart monitoring device capable of transmitting wireless heart rate data signals to a portable computing device;
(b) a processor associated with the portable computing device, the processor capable of processing heart rate data signals to determine whether the signals meet pre-established triggering criteria;
(c) a transmitter associated with the portable computing device, the transmitter capable of transmitting the triggering heart rate data signals to a wireless remote receiver/transmitter connected to a linked multi-computer network;
(d) a receiver associated with one or both of:
   (i) an emergency medical service provider; or
   (ii) at least one recipient from among a list of designated emergency contact persons,
   the receiver capable of receiving wireless/cellular transmissions via a portable computing device.

In operation, receipt of the triggering heart rate data signals by the one or both of (i) an emergency service provider or (ii) the at least one recipient provides information sufficient to determine whether an abnormality of sufficient magnitude exists to warrant an emergency medical service response to the user.

In a preferred embodiment of the foregoing medical alert system, the processor is further capable of categorizing the triggering heart rate data signals into categories for directing an emergency medical response level. The categories preferably differentiate between normal and abnormal heart rates. The categories preferably further differentiate between two abnormal heart rates, one of the heart rate categories corresponding to a heart rate below normal range and the other of the heart rate categories corresponding to a heart rate above normal range. The one category of below-normal range heart rate is preferably further differentiated between a Medical Emergency below-normal heart rate range and a Low Alert below-normal heart rate range, and the other category of above-normal range heart rate is further differentiated between a Medical Emergency above-normal heart rate range and a High Alert above-normal heart rate range.

In a further preferred embodiment, the foregoing medical alert system:
when the triggering heart rate data signals are within the Medical Emergency below-normal or within the above-normal heart rate range, the receiver provides sufficient information to both of (i) the emergency service provider and (ii) the at least one recipient to determine that an abnormality of sufficient magnitude exists to warrant an emergency medical service response to the user;
when the triggering heart rate data signals are within the Low Alert below-normal heart rate range or within the High Alert above-normal heart rate range, the receiver provides sufficient information to the at least one recipient to determine that an abnormality sufficient magnitude exists to warrant an emergency medical service response to the user.

In a further preferred embodiment of the foregoing medical alert system:
the Medical Emergency below-normal heart rate range is less than 30 beats per minute;
the Low Alert below-normal heart rate range is 30-50 beats per minute;
the High Alert above-normal heart rate range is 150-200 beats per minute; and
the Medical Emergency above-normal heart rate range is greater than 200 beats per minute.

In a further embodiment of the foregoing medical alert system, the wearable heart monitoring device transmits wireless heart rate data signals to the portable computing device using a communication protocol comprising one of a proprietary communication protocol or a near-field communication protocol. The portable computing device can be one of a smartphone or a tablet device. The linked multi-computer network is preferably the Internet. The receiver/transmitter device can be cellular-based. The cellular-based receiver/transmitter device can be a cell tower. The wireless remote receiver/transmitter connection to a linked multi-computer network can be wireless. The wireless remote receiver/transmitter connection to a linked multi-computer network can also be hard-wired. The wearable heart rate monitoring device can be wrist-worn or implanted. The wearable heart rate monitoring device can measure the user's heart rate on an ongoing basis or on an as-needed basis.

### BRIEF DESCRIPTION OF THE DRAWINGS

FIG. 1 is a schematic diagram of a medical alert systems in which a wearable heart monitor transmits heart rate data wirelessly to a smartphone, tablet or other portable computing device, which in turn broadcasts that data wirelessly to a receiver connected to the Internet, or other linked multi-computer network, to summon medical assistance and/or apprise designated recipients of the wearer's experiencing a potentially disabling cardiac event.

FIG. 2 is a flowchart of a representative embodiment of the present medical alert system.

FIG. 3 is a schematic diagram of a screen display for the Account Creation step of a software application intended to be run on the portable computing device the present medical alert system.

FIG. 4 is a schematic diagram of a screen display for the Sign-In step of a software application intended to be run on the portable computing device the present medical alert system.

FIG. 5 is a schematic diagram of a screen display for the My Profile data capturing step of a software application intended to be run on the portable computing device the present medical alert system.

FIG. 6 is a schematic diagram of a screen display for the Basic Demographics data capturing step of a software application intended to be run on the portable computing device the present medical alert system.

FIG. 7 is a schematic diagram of a screen display for the Medical Conditions data capturing step of a software application intended to be run on the portable computing device the present medical alert system.

FIG. 8 is a schematic diagram of a screen display for the Medications/Allergies data capturing step of a software application intended to be run on the portable computing device the present medical alert system.

FIG. 9 is a schematic diagram of a screen display for the My Emergency Contacts data capturing step of a software application intended to be run on the portable computing device the present medical alert system.

FIG. 10 is a schematic diagram of a screen display for the My Alert Settings data capturing step of a software application intended to be run on the portable computing device the present medical alert system.

### DETAILED DESCRIPTION OF ILLUSTRATIVE EMBODIMENT(S)

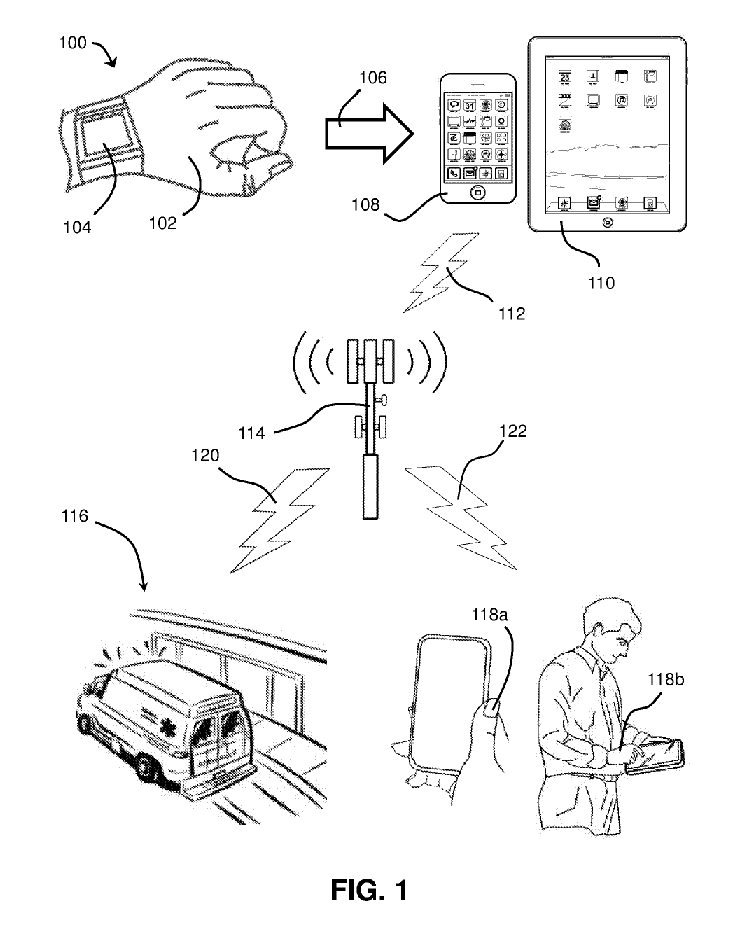

Turning first to FIG. 1, a representative medical alert system 100 is illustrated schematically. As shown, a human user 102 has a wearable heart monitoring device 104. Wearable heart monitoring device 104 transmits wireless heart rate data signals (depicted in FIG. 1 by arrow 106) to a smartphone 108, a tablet device 110, or similar hand-held or portable computing device. When the heart rate data transmitted by wearable heart monitoring device 104 is processed and determined to meet pre-established triggering criteria, one or more of smartphone 108, tablet device 110, and/or similar hand-held or portable computing device, broadcasts the user's heart rate data wirelessly (depicted in FIG. 1 by lightning bolt symbol 112) to a wireless receiver/transmitter, which is depicted in FIG. 1 by a cell tower 114.

Cell tower 114 is connected, either wirelessly or hard-wired, to the Internet or other linked multi-computer network. The connection to the Internet enables the transmission of signals indicative of the user's heart rate data to one or more of an emergency medical service (911) provider 116 and/or recipient(s) from among a designated list of emergency contact persons, one of which is depicted in FIG. 1 as a recipient 118a, who receives wireless/cellular transmissions via a smartphone, and/or a recipient 118b, who receives wireless/cellular transmissions via a tablet device. These wireless transmissions are depicted in FIG. 1 by lightning bolt symbol 120 in the case of the emergency medical service provider 116 and by lightning bolt symbol 122 in the case of recipients 118a and 118b.

Heart rate monitoring device 104 can take the form of a wrist-worn monitor, or similar wearable or implantable device, which measures the user's heart rate on an ongoing or as-needed basis. Heart rate monitor 104 can transmit signals corresponding to measured heart rate data to a mobile computing device such as smartphone 108, tablet 110, and/or similar hand-held or portable computing device. Heart rate monitoring device 104 can transmit signals via one of several wireless communications protocols, such as Bluetooth®, near-field communication, and other communication protocol that enable two electronic devices, one of which is usually a portable device such as a smartphone or tablet device, to establish communication between the devices.

The portable computing device employed in the present system receives heart rate data signals transmitted from the wearable heart rate monitoring device. The portable computing device is configured to receive and compare the received heart rate data signals according to a set of pre-established parameters to determine that the wearer (user) is experiencing, or has experienced, a cardiac event. The portable computing device can be configured via a combination of hardware and non-transitory machine readable software code that is programmed to interpret the heart rate data signals, and determine that an abnormality exists of sufficient magnitude to warrant a request for an emergency medical service response.

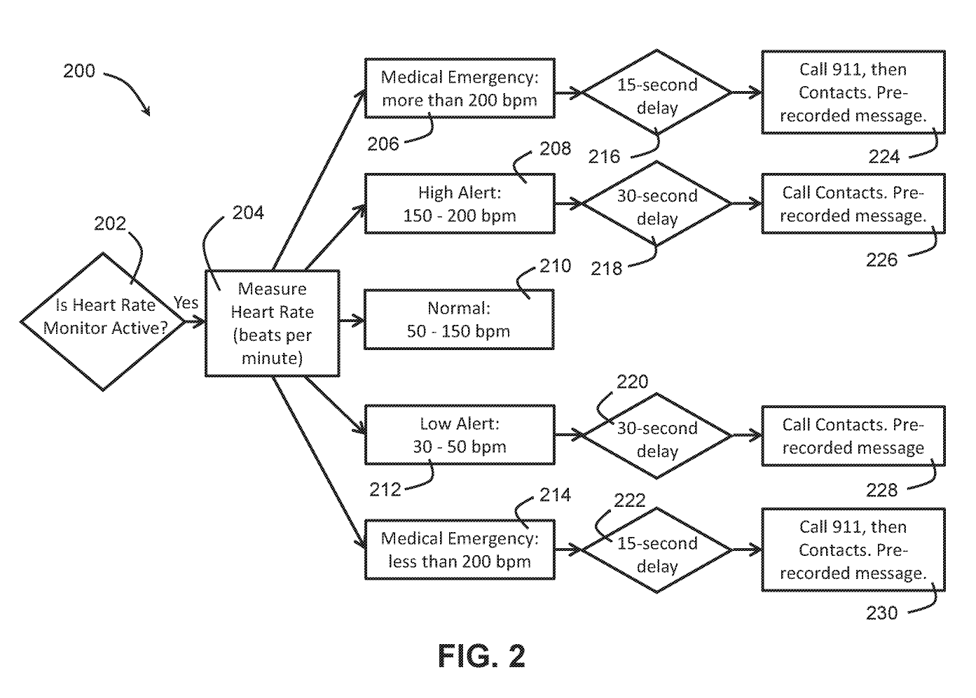

FIG. 2 is a flowchart of a representative embodiment 200 of the methodology employed in the present medical alert system. The methodology begins at step 202 with a determination as to whether the wearable heart rate monitoring device is active to detect a user's heart rate and generate data signals for transmission to a portable computing device. If the heart rate monitoring device is determined to be active, the user's heart rate is measured in beats per minute (bpm) at step 204. The measured heart rate is then screened in relation to criteria that will govern the transmission of the heart rate data signals.

As depicted in the path beginning at step 206, a heart rate greater than 200 bpm will be characterized as a Medical Emergency, in which case the system will impose a 15-second delay at step 216, which is meant to provide the user with a chance to cancel the Medical Emergency transmission if action in response to a disabling condition is not warranted. If after the 15-second delay imposed at step 216 has transpired, the system at step 224 transmits a signal, in the form of an audible and/or text message, to an emergency medical service (911) provider and then to recipient(s) from among the user's designated list of emergency contact persons.

As depicted in the path beginning at step 208, a heart rate in the range of 150-200 bpm will be characterized as a High Alert, in which case the system will impose a 30-second delay at step 208, which is meant to provide the user with a chance to cancel the High Alert transmission if not indicative of a disabling condition. If after the 30-second delay imposed at step 218 has transpired, the system at step 226 transmits a signal, in the form of an audible and/or text message, to recipient(s) from among the user's designated list of emergency contact persons.

As depicted at step 210, a heart rate in the range of 50-150 bpm will be characterized as Normal, thereby negating the transmission of a signal to either of an emergency medical service (911) provider or to recipient(s) from among the user's designated list of emergency contact persons.

As depicted in the path beginning at step 212, a heart rate in the range of 30-50 bpm will be characterized as a Low Alert, in which case the system will impose a 30-second delay at step 220, which is meant to provide the user with a chance to cancel the Low Alert transmission if not indicative of a disabling condition. If after the 30-second delay imposed at step 220 has transpired, the system at step 228 transmits a signal, in the form of an audible and/or text message, to recipient(s) from among the user's designated list of emergency contact persons.

As depicted in the path beginning at step 214, a heart rate less than 30 bpm will be characterized as a Medical Emergency, in which case the system will impose a 15-second delay at step 222, which is meant to provide the user with a chance to cancel the Medical Emergency transmission if not indicative of a disabling condition. If after the 15-second delay imposed at step 222 has transpired, the system at step 230 transmits a signal, in the form of an audible and/or text message, to an emergency medical service (911) provider and then to recipient(s) from among the user's designated list of emergency contact persons.

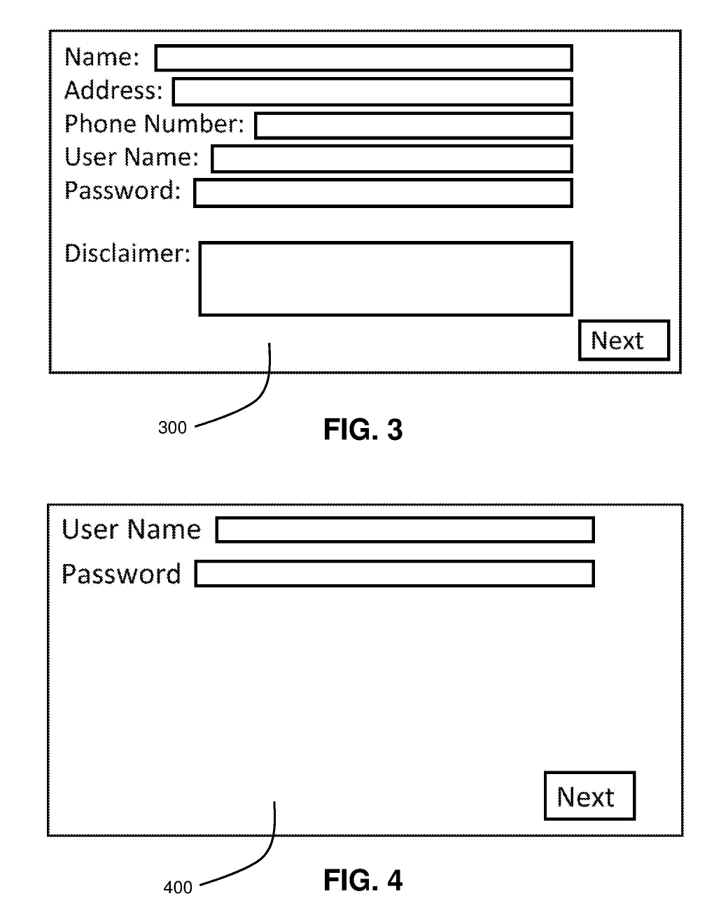

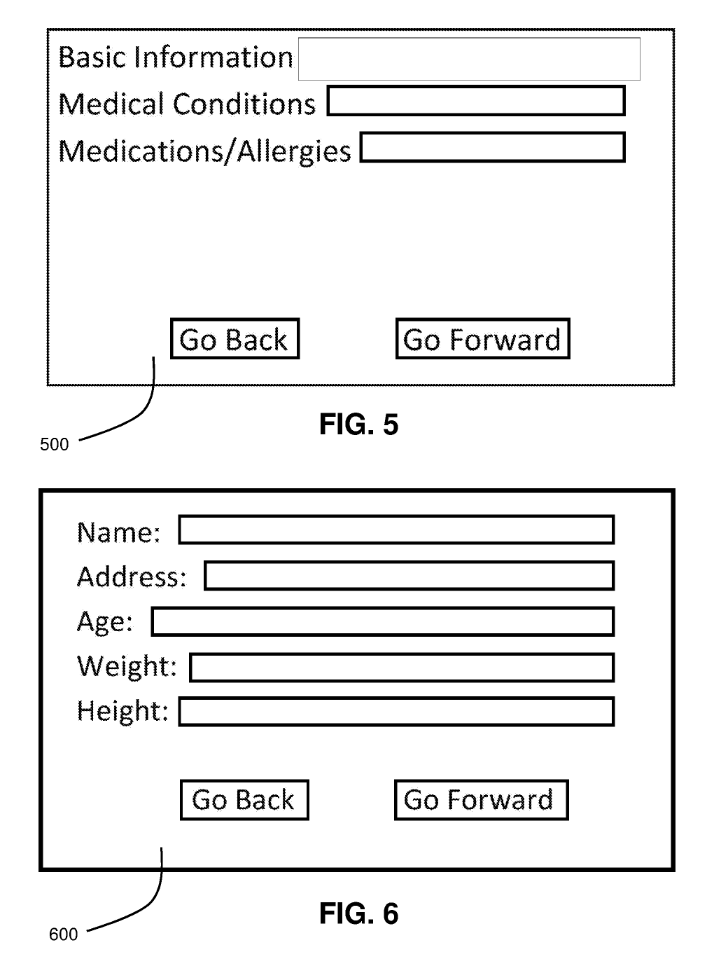

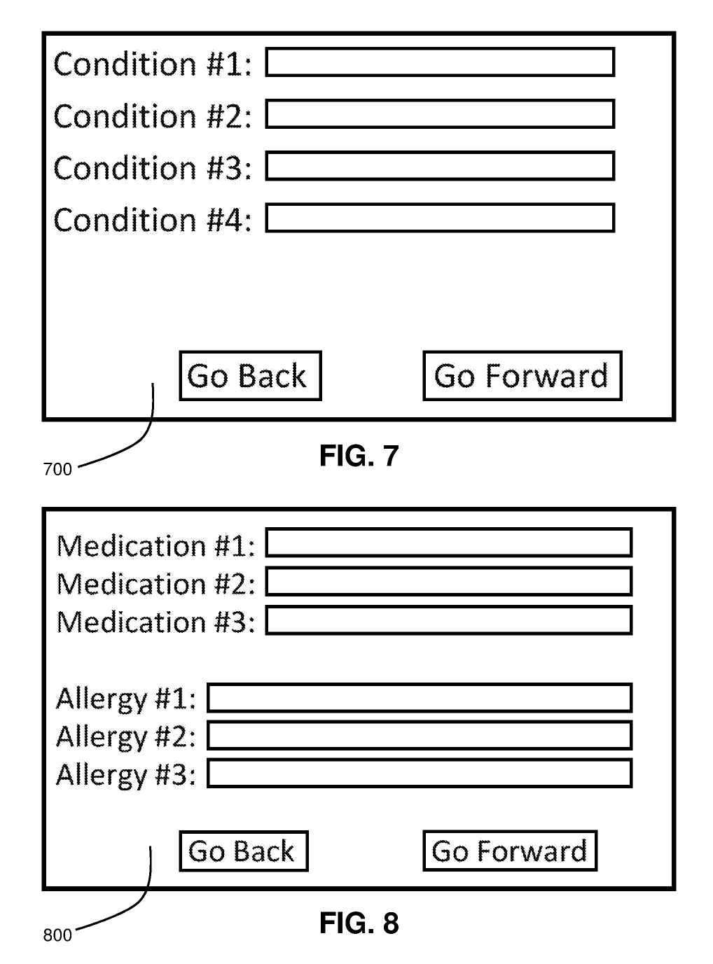

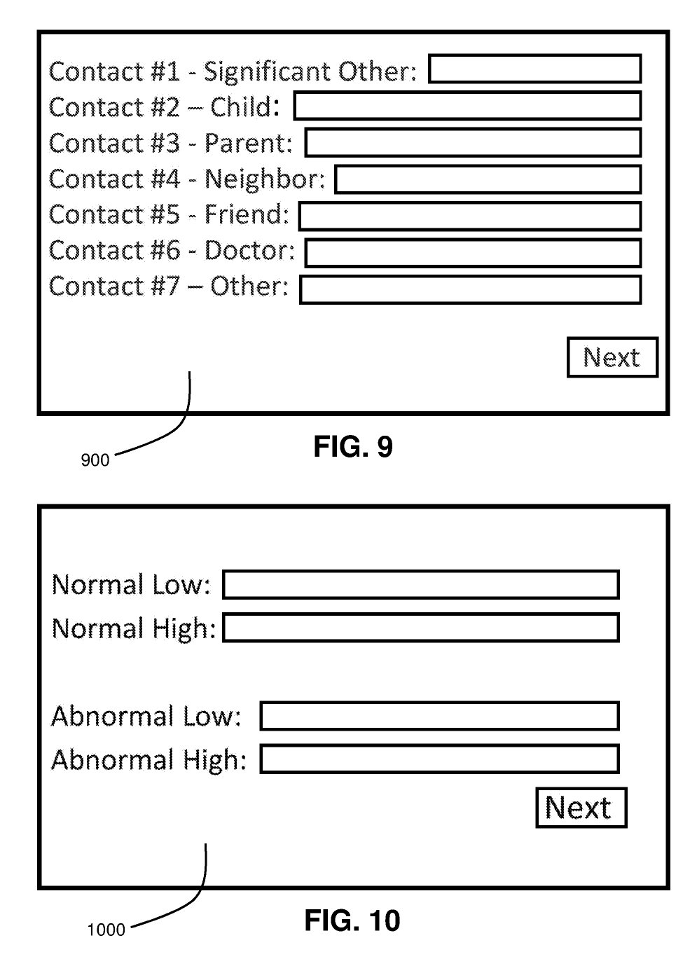

FIGS. 3-10 depict representative screen displays in a software application (app) intended to be downloaded onto the smartphone, tablet or other portable computing device associated with the present medical alert system. FIG. 3 depicts a representative screen display 300 for the Account Creation step of the illustrated app run on the portable computing device. FIG. 4 depicts a representative screen display 400 for the Sign-In step of the illustrated app. FIG. 5 depicts a representative screen display 500 for the My Profile data capturing step of the illustrated app. FIG. 6 depicts a representative screen display 600 for the Basic Demographics data capturing step of the illustrated app. FIG. 7 depicts a representative screen display for the Medical Conditions data capturing step of the illustrated app. FIG. 8 depicts a representative screen display 800 for the Medications/Allergies data capturing step of the illustrated app. FIG. 9 depicts a representative screen display for the My Emergency Contacts data capturing step of the illustrated app. FIG. 10 depicts a representative screen display for the My Alert Settings data capturing step of the illustrated app.

The heart rate monitoring device employed in the present system can be incorporated into a device that simply has the capability of place telephone calls. In this case, the software for the app can be embedded in this type of device, and calls can be initiated directly from this type of device.

The app employed in the present system can be programmed to transmit specific and detailed instructions to emergency personnel regarding health conditions of the user. The app can also be programmed to transmit the user's medication and allergy histories.

While described herein in relation to cardiac monitors, the present system could be deployed in connection with other health parameters that are capable of being monitored via wearable or implantable devices for detecting medical abnormalities. Such monitorable parameters include, for example, blood glucose and blood oxygen concentrations. The present system, although described herein in relation to human users, could also be deployed in connection with other animal species.

The present system is not described herein as being dependent upon particular computer hardware or architecture. Accordingly, the present system is not limited to any specific computer language, program or computer. It is further contemplated that the present system could be run on a stand-alone computer system or on a server computer system accessible by a plurality of client computer systems interconnected over an intranet network, or that is accessible to clients over the Internet.

While particular elements, embodiments and applications of the present invention have been shown and described, it will be understood, that the invention is not limited thereto since modifications can be made by those skilled in the art without departing from the scope of the present disclosure, particularly in light of the foregoing teachings.

What is claimed is:

1. A medical alert system comprising:
(a) a heart monitoring device wearable by a user, said wearable heart monitoring device capable of transmitting wireless heart rate data signals to a portable computing device;
(b) a processor associated with said portable computing device, said processor capable of processing heart rate data signals to determine whether said signals meet pre-established triggering criteria;
(c) a transmitter associated with said portable computing device, said transmitter capable of transmitting said triggering heart rate data signals to a wireless remote receiver/transmitter connected to a linked multi-computer network;
(d) a receiver associated with one or both of:
(i) an emergency medical service provider; or
(ii) at least one recipient from among a list of designated emergency contact persons,
said receiver capable of receiving wireless/cellular transmissions via a portable computing device,
whereby receipt of said triggering heart rate data signals by said one or both of (i) an emergency service provider or (ii) said at least one recipient provides information sufficient to determine whether an abnormality of sufficient magnitude exists to warrant an emergency medical service response to said user.

2. The medical alert system of claim 1, wherein said processor is further capable of categorizing said triggering heart rate data signals into categories for directing an emergency medical response level.

3. The medical alert system of claim 2, wherein said categories differentiate between normal and abnormal heart rates.

4. The medical alert system of claim 3, wherein said categories further differentiate between two abnormal heart rates, one of said heart rate categories corresponding to a heart rate below normal range and the other of said heart rate categories corresponding to a heart rate above normal range.

5. The medical alert system of claim 4, wherein said one category of below-normal range heart rate is further differentiated between a Medical Emergency below-normal heart rate range and a Low Alert below-normal heart rate range, and said other category of above-normal range heart rate is further differentiated between a Medical Emergency above-normal heart rate range and a High Alert above-normal heart rate range.

6. The medical alert system of claim 5, wherein:
when said triggering heart rate data signals are within said Medical Emergency below-normal or within said above-normal heart rate range, said receiver provides sufficient information to both of (i) said emergency service provider and (ii) said at least one recipient to determine that an abnormality of sufficient magnitude exists to warrant an emergency medical service response to said user;
when said triggering heart rate data signals are within said Low Alert below-normal heart rate range or within said High Alert above-normal heart rate range, said receiver provides sufficient information to said at least one recipient to determine that an abnormality sufficient magnitude exists to warrant an emergency medical service response to said user.

7. The medical alert system of claim 5, wherein:
said Medical Emergency below-normal heart rate range is less than 30 beats per minute;
said Low Alert below-normal heart rate range is 30-50 beats per minute;
said High Alert above-normal heart rate range is 150-200 beats per minute; and
said Medical Emergency above-normal heart rate range is greater than 200 beats per minute.

8. The medical alert system of claim 1, wherein said wearable heart monitoring device transmits wireless heart rate data signals to said portable computing device using a communication protocol comprising one of a proprietary communication protocol or a near-field communication protocol.

9. The medical alert system of claim 1, wherein said portable computing device is one of a smartphone or a tablet device.

10. The medical alert system of claim 1, wherein said linked multi-computer network is the Internet.

11. The medical alert system of claim 1, wherein said receiver/transmitter device is cellular-based.

12. The medical alert system of claim 11, wherein said cellular-based receiver/transmitter device is a cell tower.

13. The medical alert system of claim 1, wherein said wireless remote receiver/transmitter connection to a linked multi-computer network is wireless.

14. The medical alert system of claim 1, wherein said wireless remote receiver/transmitter connection to a linked multi-computer network is hard-wired

15. The medical alert system of claim 1, wherein said wearable heart rate monitoring device is wrist-worn or implanted.

16. The medical alert system of claim 1, wherein said wearable heart rate monitoring device measures said user's heart rate on an ongoing basis.

17. The medical alert system of claim 1, wherein said wearable heart rate monitoring device measures said user's heart rate on an as-needed basis.
